(19) United States
(12) Patent Application Publication
(10) Pub. No.: US 2022/0241079 A1
(43) Pub. Date: Aug. 4, 2022

(54) SYSTEMS AND METHODS FOR INTRAOPERATIVE BONE FUSION

(71) Applicant: Mazor Robotics Ltd., Caesarea (IL)

(72) Inventor: Yizhaq Shmayahu, Ramat HaSharon (IL)

(21) Appl. No.: 17/538,985

(22) Filed: Nov. 30, 2021

Related U.S. Application Data
(60) Provisional application No. 63/144,036, filed on Feb. 1, 2021.

(51) Int. Cl. A61F 2/30; A61F 2/44; A61F 2/46; A61L 27/38

(52) U.S. Cl. CPC A61F 2/30942; A61F 2/4455; A61F 2/4611; A61F 2002/30952; A61L 27/3821; A61F 2002/30985; A61F 2002/4632; A61L 27/3847

(57) ABSTRACT

An in-situ fusion system includes at least one robotic arm; a bioprinter; a polymerization tool; at least one processor; and a memory storing instructions for execution by the at least one processor. The instructions, when executed, cause the at least one processor to: control the at least one robotic arm to prepare at least two bone surfaces to support cellular growth; cause the bioprinter to print, from a scaffold material, a scaffold between the at least two bone surfaces; and cause the polymerization tool to induce the scaffold material to polymerize.

### CROSS REFERENCE TO RELATED APPLICATIONS

This application claims the benefit of U.S. Provisional Application No. 63/144,036, filed on Feb. 1, 2021, and entitled “Systems and Methods for Intraoperative Bone Fusion”, which application is incorporated herein by reference in its entirety.

### FIELD

The present technology generally relates to robot-assisted surgical procedures, and relates more particularly to achieving fusion of bony anatomy in a robot-assisted surgery.

### BACKGROUND

Surgical robots may assist a surgeon or other medical provider in carrying out a surgical procedure, or may complete one or more surgical procedures autonomously. Fusion procedures, whether involving the spine or elsewhere in a patient's anatomy, may be used to fix one bone or portion thereof to another bone or portion thereof.

International Patent Application No. PCT/IL2018/050384, published as WO 2018/185755 and entitled “Three Dimensional Robotic Bioprinter,” describes a minimally invasive system using a surgical robot as a three-dimensional printer for fabrication of biological tissues inside the body of a subject. The entirety of this reference is incorporated herein by reference.

### SUMMARY

Example aspects of the present disclosure include:

An in-situ fusion system, comprising: at least one robotic arm; a bioprinter; a polymerization tool; at least one processor; and a memory storing instructions for execution by the at least one processor. The instructions, when executed, cause the at least one processor to: control the at least one robotic arm to prepare at least two bone surfaces to support cellular growth; cause the bioprinter to print, from a scaffold material, a scaffold between the at least two bone surfaces; and cause the polymerization tool to induce the scaffold material to polymerize.

Any of the aspects herein, further comprising a cellular impregnation tool, wherein the memory stores additional instructions for execution by the at least one processor that, when executed, cause the at least one processor to cause the cellular impregnation tool to impregnate the scaffold with cellular elements, using a robotic arm of the at least one robotic arm to position the cellular impregnation tool.

Any of the aspects herein, wherein the cellular impregnation tool is selectively attachable to the robotic arm.

Any of the aspects herein, wherein controlling the at least one robotic arm to prepare the at least two bone surfaces to support cellular growth comprises controlling the at least one robotic arm to clean the at least two bone surfaces; and apply a surface treatment to each of the at least two bone surfaces.

Any of the aspects herein, wherein the surface treatment is a coating configured to promote adhesion of the scaffold material.

Any of the aspects herein, wherein applying the surface treatment comprises applying a surface treatment to a predetermined thickness.

Any of the aspects herein, wherein the memory stores additional instructions for execution by the at least one processor that, when executed, cause the at least one processor to: repeat the causing the bioprinter to print the scaffold and the causing the polymerization tool to induce the scaffold material to polymerize until the scaffold extends from one of the at least two bone surfaces to another of the at least two bone surfaces.

Any of the aspects herein, wherein the polymerization tool is configured to apply energy to the scaffold material to induce the scaffold material to polymerize.

Any of the aspects herein, wherein the polymerization tool is configured to apply an enzyme to the polymerization tool to induce the scaffold material to polymerize.

Any of the aspects herein, wherein the at least two bone surfaces are vertebral endplates.

Any of the aspects herein, wherein the memory stores additional instructions for execution by the at least one processor that, when executed, cause the at least one processor to insert an expandable cage between the at least two bone surfaces to hold the at least two bone surfaces in a desired position.

Any of the aspects herein, wherein the at least one robotic arm comprises a first robotic arm and a second robotic arm separate from the first robotic arm, and further wherein the first robotic arm is used to position the bioprinter for printing the scaffold and the second robotic arm is used to position the polymerization tool for inducing the scaffold material to polymerize.

Any of the aspects herein, wherein the causing the bioprinter to print a scaffold between the at least two bone surfaces and the causing the polymerization tool to induce the scaffold material to polymerize occur simultaneously.

Any of the aspects herein, wherein each of the bioprinter and the polymerization tool is selectively attachable to the at least one robotic arm.

Any of the aspects herein, wherein the at least one robotic arm comprises a single robotic arm, and further wherein the single robotic arm is used to position the bioprinter for printing the scaffold and to position the polymerization tool for inducing the scaffold material to polymerize.

A robotic surgical system comprising: a robotic arm selectively connectable to each of a preparation tool, a printing tool, and a cellular impregnation tool; at least one processor; and a memory storing instructions for execution by the at least one processor. The instructions, when executed, cause the at least one processor to: cause the robotic arm to use the preparation tool to prepare an anatomical surface inside a patient for bone growth thereon; cause the robotic arm to use the printing tool to print a scaffold inside the patient that connects to the anatomical surface; and cause the robotic arm to use the cellular impregnation tool to impregnate the scaffold with bone tissue cells.

Any of the aspects herein, wherein the scaffold is printed from a scaffold material, and further wherein the memory stores additional instructions for execution by the at least one processor that, when executed, further cause the at least one processor to: cause the robotic arm to use a polymerization tool to induce polymerization of the scaffold material.

Any of the aspects herein, wherein preparing the anatomical surface comprises causing the robotic arm to use the preparation tool to create a plurality of holes in the anatomical surface.

Any of the aspects herein, wherein the scaffold is printed and impregnated with bone tissue cells one layer at a time.

Any of the aspects herein, wherein the anatomical surface is a vertebral endplate; the scaffold, when finished, connects the vertebral endplate with an opposite vertebral endplate; and a first layer of the scaffold is printed on an anterior ligament.

Any of the aspects herein, wherein impregnating the scaffold with bone tissue cells comprises filling a volume defined by the scaffold with bone tissue cells.

Any of the aspects herein, further comprising an imaging device, and wherein the memory stores additional instructions for execution by the at least one processor that, when executed, further cause the at least one processor to: cause the imaging device to capture an image of the anatomical surface after the anatomical surface has been prepared for bone growth thereon.

An in-situ vertebral fusion method comprising: controlling a 3D printer, operably connected to a robotic arm, to print, in between two vertebral endplates and using a polymerizable scaffold material, a scaffold structure; and controlling a polymerization tool, operably connected to the robotic arm, to induce polymerization of the scaffold material.

Any of the aspects herein, further comprising: controlling an impregnation tool, operably connected to the robotic arm, to impregnate the scaffold structure with bone growth tissue.

Any of the aspects herein, further comprising: controlling the robotic arm, operably connected to an endplate preparation tool, to prepare each of the two vertebral endplates for bone growth thereon.

Any of the aspects herein, wherein controlling the robotic arm to prepare each of the two vertebral endplates for bone growth thereon comprises controlling the robotic arm to clean each of the two vertebral endplates and to apply a surface treatment to each of the two vertebral endplates.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Z₀).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure.

FIGS. 2A to 2I illustrate various steps of a vertebral fusion process according to at least one embodiment of the present disclosure. More specifically:

FIG. 2A illustrates a pair of vertebrae to be fused;

FIG. 2B illustrates the pair of vertebrae of FIG. 2A, following removal of the intervertebral disc;

FIG. 2C illustrates the pair of vertebrae of FIG. 2A, following expansion of the intervertebral space;

FIG. 2D illustrates preparation of an endplate of one of the pair of vertebrae of FIG. 2A;

FIG. 2E illustrates further preparation of an endplate of one of the pair of vertebrae of FIG. 2A;

FIG. 2F illustrates in-situ printing of a scaffold in the intervertebral space of the pair of vertebrae of FIG. 2A;

FIG. 2G illustrates polymerization of the scaffold material that comprises the scaffold between the pair of vertebrae of FIG. 2A;

FIG. 2H illustrates impregnation, with cellular elements, of the scaffold between the pair of vertebrae of FIG. 2A; and

FIG. 2I illustrates a completed intervertebral fusion of the pair of vertebrae of FIG. 2A.

FIG. 3 is a flowchart according to at least one embodiment of the present disclosure.

FIG. 4 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10× Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

Spinal fusion is a major component of surgical solutions for various degenerative, deformative, traumatic and other spinal conditions. A fusion may employ allograft, autograft, and/or synthetic bone or bone-like materials, sometimes along with bone growth inducing materials, to promote fusion between adjacent vertebrae or between pelvic bones. The process of bony fusion with these methods may take months to complete. Hence, a metal construct, perhaps involving rods and screws, may be used to provide internal fixation during the recovery period. Establishing the internal fixation construct adds time, cost, and risk to the surgical procedure. Historically, external fixation was used, but required prolonged bed rest, carrying its own risks. The delayed fusion also implies that in the event of non-fusion, revision surgery might be required.

The current invention supports intra-operative fusion, alleviating the need for post-operative fixation and enabling intra-operative monitoring of fusion extent.

Bone bioprinting is currently used to grow bone elements in the lab for future implantation and/or to fill bone defects—for example, after tumor resection and trauma.

Embodiments of the present disclosure involve in-situ printing of a polymeric scaffold, which is then embedded with bone tissue cells. The scaffold material can be induced to polymerize after printing in various ways, including using one or more enzymes and/or applying light energy. Polymerization may also be induced using remote energy sources like focused ultrasound. After polymerization, the printed scaffold has significant strength, that can be sufficient for the temporary fixation needed during cellular growth.

Fusion techniques according to embodiments of the present disclosure include one or more of: 1) robotic end plate (for interbody fusion) or other surface preparation, which may comprise removing disc material or other soft tissue remnants, conditioning the end plate(s) or other surface to support bony growth, facet decortication, and/or cartilage removal; 2) robotic injection of the scaffold polymer; 3) robotic induction of polymerization using external energy sources; and/or 4) robotic impregnation of the scaffold with the needed cellular elements.

The process may be performed in a layered fashion, with multiple repeats of steps 2-4.

Embodiments of the present disclosure may be used for fusion of vertebrae, a sacro-iliac joint, a facet joint, and/or pieces of a broken large bone. Stated differently, embodiments of the present disclosure may be used, for example, for vertebral/interbody fusion, articular fusion, sacroiliac joint fusion, and repair of long bone fractures (including, e.g., hip fractures).

Embodiments of the present disclosure provide technical solutions to one or more of the problems of (1) achieving fusion of two bony anatomy elements; (2) reducing patient recovery time following a fusion procedure; (3) reducing the number of implants required to achieve fusion; (4) achieving fusion without implanting rods, screws, or metal into a patient's body; and (5) reducing a need for pre-manufactured implants to achieve fusion.

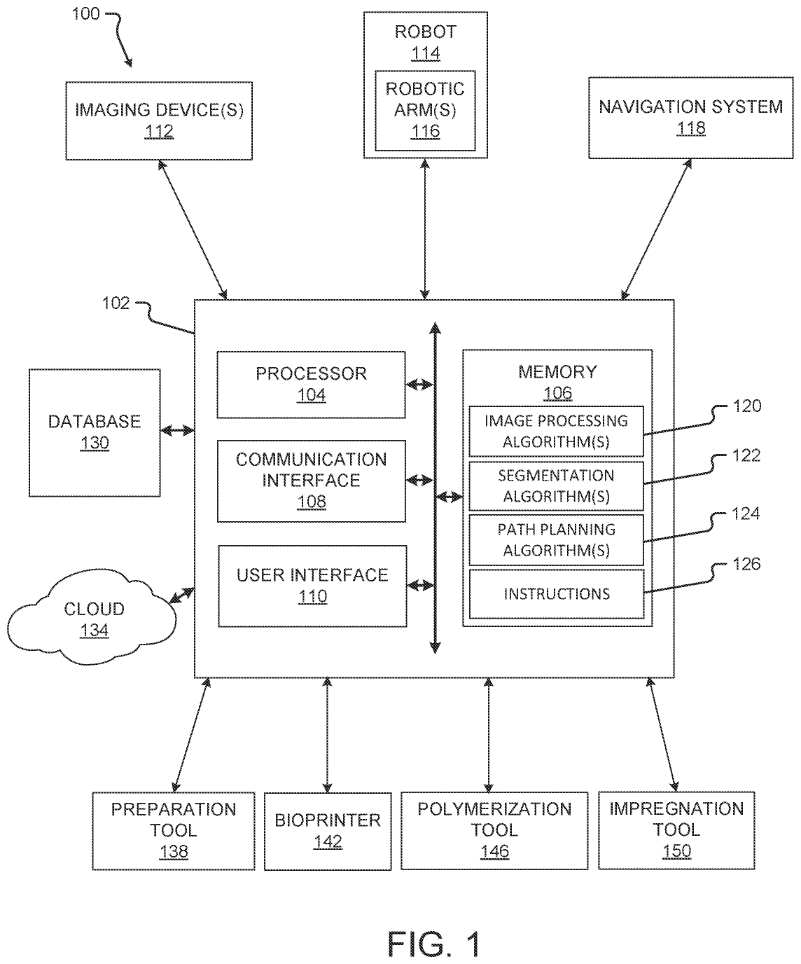

Turning first to FIG. 1, a block diagram of a system 100 according to at least one embodiment of the present disclosure is shown. The system 100 may be used for intraoperative bone fusion according to embodiments of the present disclosure, and/or carry out one or more other aspects of one or more of the methods disclosed herein. The system 100 comprises a computing device 102, one or more imaging devices 112, a robot 114, a navigation system 118, a database 130, a cloud or other network 134, a preparation tool 138, a bioprinter 142, a polymerization tool 146, and an impregnation tool 150. Systems according to other embodiments of the present disclosure may comprise more or fewer components than the system 100. For example, the system 100 may not include the imaging device 112, the robot 114, the navigation system 118, one or more components of the computing device 102, the database 130, the cloud 134, the preparation tool 138, the bioprinter 142, the polymerization tool 146, and/or the impregnation tool 150.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions 126 stored in the memory 106, which instructions 126 may cause the processor 104 to carry out one or more computing steps utilizing or based on data received from or via the imaging device 112, the robot 114, the navigation system 118, the database 130, the cloud 134, the preparation tool 138, the bioprinter 142, the polymerization tool 146, and/or the impregnation tool 150.

The memory 106 may be or comprise RAM, DRAM, SDRAM, other solid-state memory, any memory described herein, or any other tangible, non-transitory memory for storing computer-readable data and/or instructions (e.g., instructions 126). The memory 106 may store information or data useful for completing, for example, any step of the methods 300 and/or 400 described herein, or of any other methods. The memory 106 may store, for example, one or more image processing algorithms 120, one or more segmentation algorithms 122, one or more path planning algorithms 124, and/or instructions 126. Such instructions or algorithms may, in some embodiments, be organized into one or more applications, modules, packages, layers, or engines. The algorithms and/or instructions may cause the processor 104 to manipulate data stored in the memory 106 and/or received from or via the imaging device 112, the robot 114, the database 130, the cloud 134, the preparation tool 138, the bioprinter 142, the polymerization tool 146, and/or the impregnation tool 150.

The computing device 102 may also comprise a communication interface 108. The communication interface 108 may be used for receiving image data or other information from an external source (such as the imaging device 112, the robot 114, the navigation system 118, the database 130, the cloud 134, the preparation tool 138, the bioprinter 142, the polymerization tool 146, the impregnation tool 150, and/or any other system or component not part of the system 100), and/or for transmitting instructions, images, or other information to an external system or device (e.g., another computing device 102, the imaging device 112, the robot 114, the navigation system 118, the database 130, the cloud 134, the preparation tool 138, the bioprinter 142, the polymerization tool 146, the impregnation tool 150, and/or any other system or component not part of the system 100). The communication interface 108 may comprise one or more wired interfaces (e.g., a USB port, an ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, intervertebral disc, veins, tissue, intervertebral space, etc.) and/or other aspects of patient anatomy to yield image data (e.g., image data depicting or corresponding to a bone, intervertebral disc, veins, tissue, intervertebral space, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position, orient, and/or operate one or more of the imaging device 112, the preparation tool 138, the bioprinter 142, the polymerization tool 146, the impregnation tool 150, and/or any other object at one or more precise position(s) and orientation(s), and/or to return the one or more objects to the same position(s) and orientation(s) at a later point in time. The robot 114 may additionally or alternatively be configured to manipulate and/or operate any surgical tool described herein and/or any other surgical tool (whether based on guidance from the navigation system 118 or not) to accomplish or to assist with a surgical task. In some embodiments, the robot 114 (and more specifically, the robotic arm 116) may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm(s) 116 (as well as any object or element held by or secured to the robotic arm), and/or that facilitate operation of a surgical tool held by the robotic arm(s) 116.

In some embodiments, reference markers (i.e., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system may comprise one or more electromagnetic sensors. In various embodiments, the navigation system 118 may be used to track a position and orientation (i.e., pose) of the imaging device 112, the robot 114 and/or robotic arm 116, the preparation tool 138, the bioprinter 142, the polymerization tool 146, the impregnation tool 150, and/or one or more other objects (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The preparation tool 138 may be or include any one or more tools useful for preparing a vertebral endplate or other anatomical surface for fusion according to embodiments of the present disclosure. In some embodiments, such preparation may include cleaning the endplate or other surface of cartilage, soft tissue, or other matter (e.g., to remove material that might prevent the growth of blood vessels and/or the passage of nutrients into growing bone); may enable the endplate or other surface to adhere to a printed scaffold structure (or vice versa); may enable, stimulate, and/or facilitate cellular growth (e.g., growth of bone tissue cells or other cells); and/or may strengthen or otherwise prepare the endplate or other surface to be fused in accordance with embodiments of the present disclosure. Accordingly, the preparation tool 138 may be or comprise a scraper, a knife, a brush, tweezers, a clamp, a gripper, a vacuum (for suctioning debris), a sprayer (e.g., for spraying a washing fluid, or for spraying a chemical or other coating onto the endplate or other surface), a spiked roller (e.g., for stimulating bleeding of the endplate or other bone surface, and/or to facilitate vessel growth within the printed or otherwise deposited material or tissue); an applicator (e.g., for applying a controlled-thickness layer of a chemical or other material on a surface); and/or any other surface preparation tool.

The preparation tool 138 may be or comprise one or more active tools (e.g., powered tools that are motorized or otherwise actuated) and/or one or more passive tools (e.g., unpowered tools that lack any internal actuator. The preparation tool 138 may be or comprise one or more smart tools (e.g., one or more tools comprising a processor or other device that controls one or more operating characteristics or functions of the tool) and/or one or more tools that lack such processing capability. The preparation tool 138 may be configured to convert one form of energy to another (e.g., to convert electrical energy into mechanical energy via one or more actuators), and/or to provide an interface between a robotic arm 116 (or, in some embodiments, a human user) and a vertebral endplate or other surface to be fused. The preparation tool 138 may be configured for manual use and/or for connection to and/or manipulation thereof by a robotic arm 116.

In some embodiments, the preparation tool 138 may be configured to utilize a fluid to facilitate the disk preparation process. For example, the preparation tool 138 may be configured to spray water or saline onto a surface to dislodge one or more particles from the surface. In such embodiments, the preparation tool 138 may comprise an internal fluid reservoir, and/or may comprise an inlet for receiving the fluid from an external reservoir. The preparation tool 138 may additionally or alternatively comprise a vacuum source (or be connectable to a vacuum source), which may enable the preparation tool 138 to apply suction to the anatomical surface (or elsewhere) to assist in removing anatomical tissue, fluids, and/or other material from an anatomical surface or volume. The preparation tool 138 may additionally or alternatively be or comprise a powered cutting, scraping, brushing, and/or polishing tool.

The bioprinter 142 is a 3D printer (whether standing alone or as held and/or controlled by a robotic arm 116) configured to print using a bioink. A “bioink,” as used herein, is any ink usable by a 3D printer that utilizes natural materials, synthetic materials, and/or a combination thereof and that is biocompatible. Bioinks used herein may comprise collagen and/or other materials that are found in a natural disc. Such materials may be printed within an interbody cavity in a dissolved form, then polymerized in situ as described elsewhere herein. The bioprinter may be held (or otherwise supported) and manipulated by a robotic arm 116, and may be used in conjunction with a robotic arm 116 to print a scaffold or other structure (from bioink) in-situ (e.g., between two bones in a human body that need to be fused). In some embodiments, the bioink may be polymerizable. In other words, subjection of the bioink to one or more enzymes, chemicals, and/or types of energy may cause the bioink to polymerize. In some embodiments, polymerization of the bioink may cause the bioink to harden and/or otherwise impart material properties to the bioink that are favorable for fusing two bones or other anatomical elements together. The bioprinter 142 may comprise an internal bioink reservoir, and/or may comprise an inlet for receiving the bioink from an external reservoir.

The polymerization tool 146 is a tool configured to induce polymerization of a bioink. The polymerization tool 146 may be configured to spray or otherwise apply an enzyme and/or chemical onto the printed bioink (e.g., a scaffold or other structure, or portion thereof) to induce polymerization thereof. In such embodiments, the polymerization tool may comprise a reservoir of the enzyme and/or chemical, and/or may simply comprise an inlet for receiving the enzyme and/or chemical from an external reservoir.

The polymerization tool 146 may additionally or alternatively be or comprise an energy delivery device, configured to deliver light energy, ultrasound energy, and/or any other energy form that will induce polymerization in the printed bioink. The polymerization tool 146 may be configured to deliver a focused ray of energy, so that only a very small amount or volume (or a very precise amount or volume, regardless of quantity or size) of bioink is induced to polymerize at once. In some embodiments, the polymerization tool 146 may comprise a changeable lens, aperture, or other device that enables energy to be emitted from the polymerization tool 146 in various shapes and/or patterns. For example, in some embodiments, the polymerization tool 146 may be configured to deliver energy to a single point, or along a line, or over an area, or through a particular volume. In some embodiments, robotic manipulation of the polymerization tool 146 may be utilized to achieve a high degree of spatial accuracy of delivered energy, so as to ensure that polymerization of the printed bioink occurs only in precise locations where the polymerization is desired.

The particular polymerization tool 146 used in an embodiment of the present disclosure may be selected, for example, based on the type of bioink selected, and/or vice versa. In some embodiments, a polymerization tool 146 configured to deliver energy for the purpose of inducing polymerization may enable more precise control over where polymerization occurs and where it does not than may be possible with a polymerization tool 146 configured to deliver an enzyme or chemical to induce polymerization of the bioink.

The impregnation tool 150 may be any tool configured to deliver cellular elements—e.g., bony cells, bone growth tissue, allograft, autograft—for impregnation of a polymerized scaffold structure or portion thereof printed using bioink. In some embodiments, the impregnation tool 150 comprises a reservoir for storing and/or an inlet for receiving cellular elements; an outlet from which cellular elements may be injected or otherwise discharged; and a pumping or conveyance system configured to move the cellular elements from the reservoir and/or inlet to the outlet. The impregnation tool 150 may be configured to deliver cellular elements into a scaffold structure or portion thereof at any pressure greater than or equal to atmospheric pressure.

The impregnation tool 150 may also be a printer or a printer-like device, and may comprise a printing head similar to that of a more traditional inkjet printer. In such embodiments, the impregnation tool 150 may be configured to “print” cellular elements one layer at a time, in an iterative fashion with the printing and polymerization of individual layers of a bioink scaffold structure (as further described below).

Like the preparation tool 138, the bioprinter 142, and the polymerization tool 146, the impregnation tool 150 is configured to be secured to or otherwise held by, manipulated by, and/or operated by a robotic arm 116. In some embodiments, the preparation tool 138, the bioprinter 142, the polymerization tool 146, and/or the impregnation tool 150 may be configured for manual operation while being supported or held by a robotic arm; for automatic operation while be supported or held manually; and/or for purely manual support and operation.

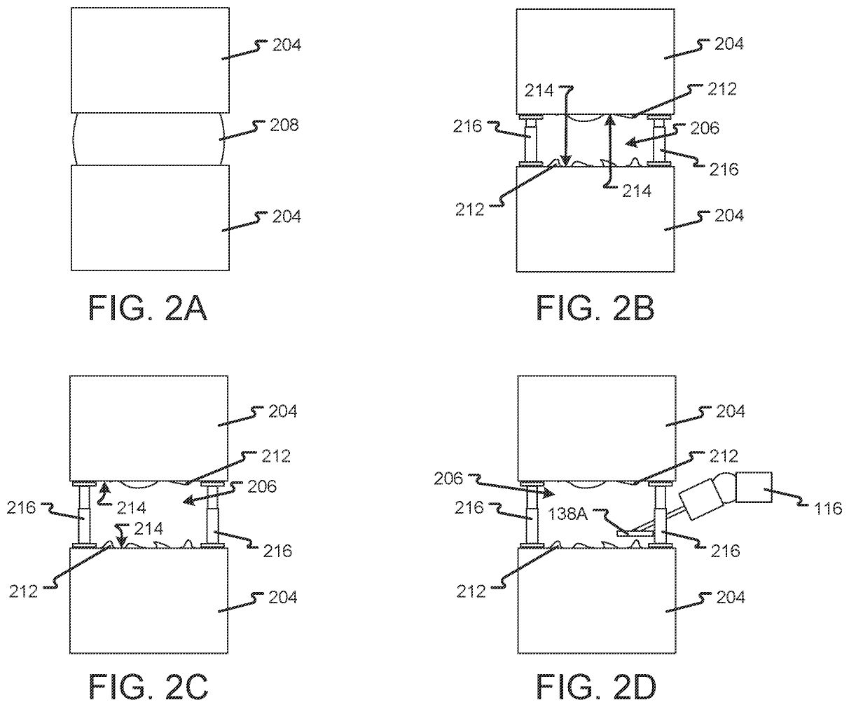

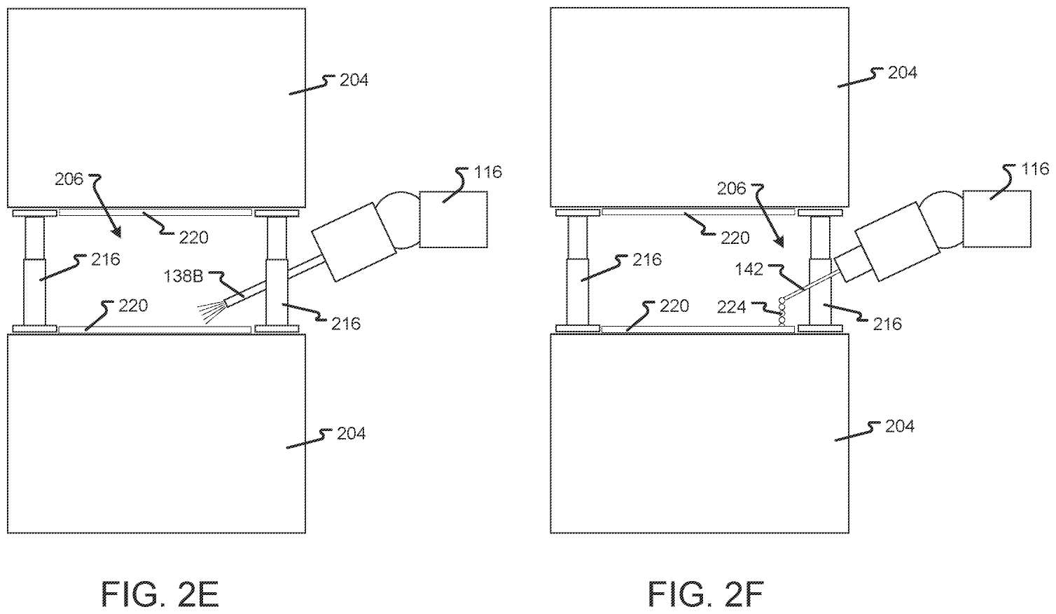

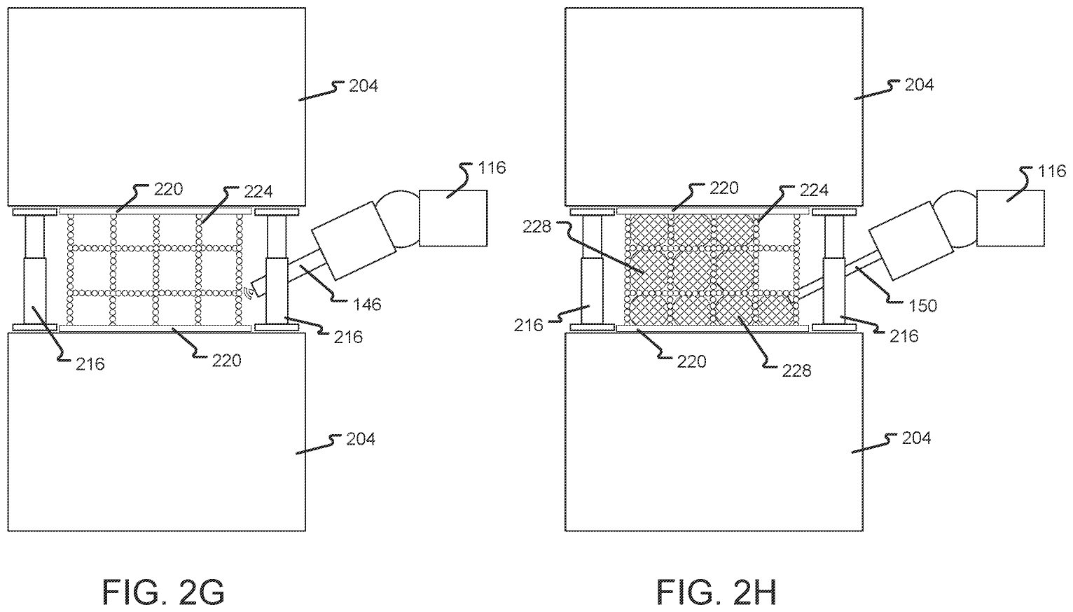

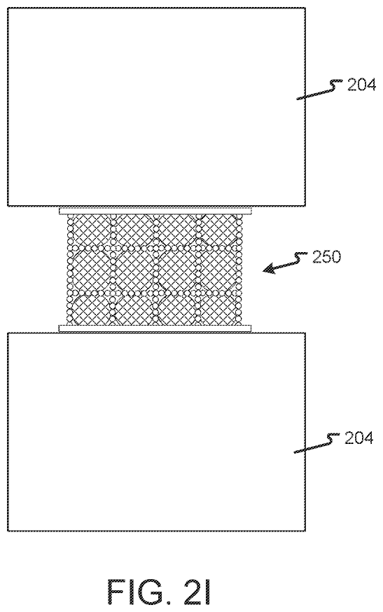

The system 100 or similar systems may be used, for example, to carry out one or more aspects of the process described in connection with the FIGS. 2A-2I, and/or of one or both of the methods 300 and/or 400 described herein. The system 100 or similar systems may also be used for other purposes.

FIGS. 2A-2I illustrate various steps of a fusion process according to at least one embodiment of the present disclosure. Elements identified in one or more of FIGS. 2A-21 may not be identified in one or more others of FIGS. 2A-21 to avoid unnecessary crowding of the figures.

FIG. 2A shows a pair of adjacent vertebrae 204 having an intervertebral disc 208 occupying the intervertebral space therebetween. Whether due to existing damage to the disc 208, and/or one or both of the vertebrae 204, the pair of adjacent vertebrae 204 need to be fused.

FIG. 2B shows the pair of vertebrae 204 with the intervertebral disc 208 removed from the intervertebral space therebetween. One or more expandable cages or other spacing tools 216 have been inserted into the intervertebral space 206. One or more disc remnants, pieces of cartilage, or other soft tissue debris 212 remains attached to the vertebral endplates 214.

FIG. 2C shows the two vertebrae 204 with an expanded intervertebral space 206 therebetween due to expansion of the expandable cages or other spacing tools 216.

In FIG. 2D, a robotic arm 116 is being used to introduce a first preparation tool 138A into the intervertebral space 206, where the robotic arm 116 may then manipulate the first preparation tool 138A to remove the disc remnants, pieces of cartilage, or other soft tissue debris 212. The preparation tool 138 may comprise, for example, a scrub brush, one or more cutting elements, a scraper, and/or any other device for removing the disc remnants, pieces of cartilage, or other soft tissue debris 212 from the endplates 214.

In FIG. 2E, the disc remnants, pieces of cartilage, or other soft tissue debris 212 have been removed from the endplates 214, and the robotic arm 116, now equipped with a second preparation tool 138B, has applied/is applying a coating 220 to each of the endplates 214. In some embodiments of the present disclosure, preparation of a surface for fusion thereof may require that a chemical or other material coating 220 is applied to the surface. Such a coating 220 may, for example, facilitate adhesion of bioink to the surface (e.g., for printing a scaffolding thereon or connected thereto); promote growth of bony tissue on the surface; or otherwise improve a likelihood of success of a fusion procedure.

The preparation tool 138B may be or comprise a sprayer, a roller, or any other applicator suitable for applying the coating 220 to the endplates 214. In some embodiments, the preparation tool 138B may be configured to apply a coating 220 having a precise thickness (e.g., of 50 to 100 microns, or of 100 to 200 microns, or of 200 to 300 microns, or of 300 to 500 microns, or of 500 to 1000 microns, or 1000; and with tolerances of, for example, less than 500 microns, or less than 250 microns, or less than 100 microns, or less than 50 microns, or less than 20 microns, or less than 10 microns). Also in some embodiments, the preparation tool 138B may be configured to apply a coating 220 having a line width of 100 to 200 microns, or 200 to 300 microns, or 300 to 400 microns, with an alignment error of 5 to 10 microns, or 10 to 20 microns, or 20 to 30 microns, or 30 to 40 microns. The coating 220 may require a tolerance that is not manually achievable, and therefore that can only be achieved using the robotic arm 116 and the preparation tool 138B.

In FIG. 2F, the robotic arm 116 is using the bioprinter 142 to print a scaffolding structure 224 out of bioink on the coating 220. In embodiments where no coating 220 is applied, a portion of the scaffolding structure may be printed directly on the endplate 214 or other anatomical surface. Also, in some embodiments, the scaffolding structure may be printed on an anterior ligament or other surface that defines an edge of the intervertebral space, and may be extended in the direction of both endplates 214 before being attached thereto.

FIG. 2G shows a completed scaffolding structure 224 extending through the intervertebral space 206 from one endplate 214 to the other endplate 214. Although FIG. 2G shows only a two-dimensional view of the scaffolding structure 224, that structure 224 extends throughout the intervertebral space 206 in three dimensions. The robotic arm 116 is manipulating the polymerization tool 146 to induce polymerization of the bioink that forms the scaffolding structure 224. The polymerization tool 146 may be emitting a focused light beam (e.g., a laser) and/or ultrasound for the purpose of inducing polymerization of the bioink scaffolding structure. In other embodiments, the polymerization tool 146 may emit another kind of energy. In some embodiments, a focused beam of ultrasound—generated by an ultrasound emitter positioned external to the patient—may be used to bathe the scaffolding structure 224 in ultrasound energy and induce polymerization thereof. In such embodiments, the robotic arm 116 may or may not be used to manipulate the polymerization tool 146.

Energy (or enzymes, chemicals, or any other polymerization-inducing agent) may be carefully emitted by the polymerization tool 146 (which may in turn be carefully controlled by the robotic arm 116) so as to induce polymerization only of scaffolding structure 224 within the boundaries of the intervertebral space 206 or other predetermined boundaries. In other words, if any bioink is printed or otherwise introduced into a volume that the scaffolding structure 224 is not intended to occupy, any such bioink may not be induced to polymerize. Once the desired scaffolding structure 224 has been polymerized, any remaining non-polymerized bioink may be washed away, suctioned, or otherwise removed from the patient's body.

Although FIGS. 2F-2G illustrate a scaffolding structure 224 created by additive manufacturing (e.g., 3D printing), in some embodiments a scaffolding structure may be generated by filling an entirety of the intervertebral space 206 with a bioink, then using a polymerization tool 146 to induce polymerization of a scaffolding structure within the volume of bioink. The non-polymerized bioink may then be washed away, suctioned, or otherwise removed from the intervertebral space, leaving only the polymerized scaffolding structure 224.

Additionally, although FIGS. 2F and 2G illustrate the completion of a scaffolding structure 224 prior to polymerization of any portion thereof using a polymerization tool 146, embodiments of the present disclosure encompass the iterative and/or simultaneous completion of these two steps. In other words, in some embodiments, a first layer of bioink may be printed using a bioprinter 142, after which that layer of bioink may be induced to polymerize using the polymerization tool 146. A second layer of bioink may then be printed and induced to polymerize, and so forth until the entire scaffolding structure is complete.

In still other embodiments of the present disclosure, a first robotic arm 116 may support a bioprinter 142 and be controlled to print the scaffolding structure 224, and a second robotic arm 116 may support a polymerization tool 146 and be controlled to induce polymerization of the just-printed bioink. In these embodiments, printing and polymerization of the scaffolding structure may occur simultaneously or near simultaneously.

Although the scaffolding structure 224 in FIGS. 2F and 2G is shown as having a grid pattern, the scaffolding structure 224 in other embodiments of the present disclosure may be printed in any three-dimensional pattern. The scaffolding structure 224 may comprise one or more of linear elements, curved elements, intertwined elements, flat surfaces, curved surfaces, and/or any other elements. Once completed, the scaffolding structure may occupy less than 50%, or less than 40%, or less than 30%, or less than 20%, or less than 10%, or less than 5% of intervertebral space 206.

FIG. 2H illustrates a robotic arm 116 using an impregnation tool 150 to impregnate the scaffolding structure 224 with cellular elements 228. The cellular elements 228 may be or comprise, for example, bone tissue cells, allograft, autograft, and/or any other material useful for growing bone. As with the steps illustrated in FIGS. 2F and 2G, impregnation of the scaffolding structure 224 with cellular elements 228 may occur in an iterative fashion (e.g., with the impregnation tool 150 being used to impregnate one layer of polymerized scaffolding at a time, prior to the next layer of the scaffolding being printed), or simultaneously with printing of the scaffolding structure 224 and polymerization thereof. In the latter instance, a bioprinter 142 may be used to continuously print the various elements of the scaffolding structure 224, the polymerization tool 146 may be used to induce polymerization of the bioink shortly after the printing thereof, and the impregnation tool 150 may be used to impregnate portions of polymerized scaffolding structure 224.

In some embodiments, the scaffolding structure 224 may define the outer limits of the volume filled by the cellular elements 228. In other embodiments, the cellular elements 228 may extend beyond an outer perimeter of the scaffolding structure 224.

With reference now to FIG. 21, once impregnation of the scaffolding structure 224 with cellular elements 228 is complete, the expandable cages or other spacing tools 216 may be removed from the intervertebral space 206. The intervertebral structure 250 may be sufficiently strong to withstand the forces expected to be exerted thereon (e.g., due to normal patient activity) immediately. In still other embodiments, the intervertebral structure 250 may be sufficiently strong to withstand forces expected to be exerted thereon within less than fifteen minutes, or less than thirty minutes, or less than one hour, or less than two hours, or less than three hours, or less than four hours, or less than five hours after completion of the intervertebral structure 224. As a result, patients undergoing fusion procedures according to embodiments of the present disclosure may be able to resume normal activity in a matter of hours, rather than undergoing a multi-day recovery such as might be associated with fusion methods involving implantation of one or more intervertebral bodies, a plurality of pedicle screws, and/or one or more rods.

In some embodiments, the expandable cages or other spacing tools 216 may be single-use, disposable tools, in which case they may (but need not) be cut away from or otherwise destructively removed from the intervertebral space 206. In other embodiments, the expandable cages or other spacing tools 216 are re-useable. Any expandable cage or other spacing tool may be used in connection with fusion methods according to embodiments of the present disclosure.

Over time, the cellular elements 228 of the intervertebral structure 250 will result in bone growth in the intervertebral space 206, such that the vertebrae 204 will eventually be fused by bone. As that bone growth occurs, the intervertebral structure 250 provides significant fixation of the spine, which in some embodiments is sufficient to enable normal (e.g., non-strenuous) patient activity. The ability to provide such fixation without requiring implantation of one or more intervertebral bodies, pedicle screws, and/or rods represents a significant advance in spinal fusion surgery, associated with beneficial effects including reduced fusion times (e.g., on the order of days or weeks, down from months), reduced patient trauma, reduced patient recovery times, reduced need for subsequent revision surgeries (e.g., due to non-fusion), reduced limitations on post-operative patient mobility, and improved outcomes.

In each embodiment of the present disclosure, a surgical plan may be used to guide each step of the fusion process, including, for example, preparation of the anatomical surface(s) using a preparation tool such as the preparation tool 138, printing of the scaffold using a bioprinter such as the bioprinter 142, polymerization of the printed scaffold using a polymerization tool 146, and/or impregnation of the polymerized scaffold using an impregnation tool such as the impregnation tool 150. The surgical plan may define, for example, a design of the scaffold, how the scaffold will be positioned within a given in situ volume, where the printing of the scaffold will begin, which portions of the scaffold will be printed in what order, and/or how if at all the printing, polymerization, and/or impregnation processes will be combined (e.g., whether printing, polymerization, and impregnation will occur sequentially, or will be iterated for successive layers of the scaffold, or will be conducted simultaneously). Any robotic arm described herein may be controlled, in some embodiments of the present disclosure, based in whole or in part on such a surgical plan, which may be stored in and/or retrieved from or via a memory such as the memory 106, a database such as the database 130, a network such as the cloud 134, and/or any other component of a system such as the system 100. In other embodiments, any such robotic arm may be controlled, in whole or in part, manually and/or based on navigation or other guidance.

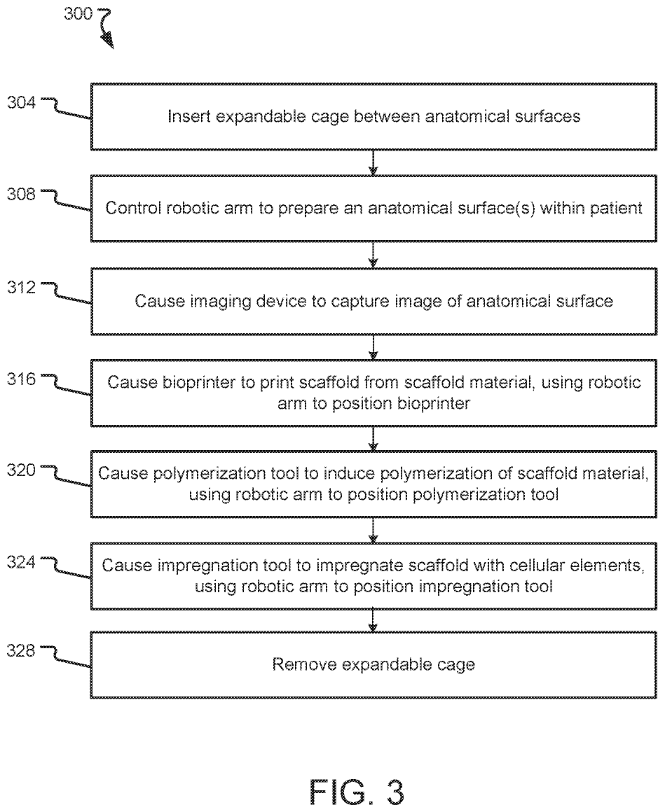

FIG. 3 depicts a method 300 that may be used, for example, to achieve fusion of two anatomical surfaces such as vertebral endplates or other bony anatomy. One or more aspects of the method 300 may be used independently and/or together with one or more aspects of any other method described herein according to embodiments of the present disclosure.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing instructions (e.g., instructions 126) stored in a memory such as the memory 106. The instructions may correspond to one or more steps of the method 300 described below. The instructions may cause the processor to execute one or more algorithms, such as an image processing algorithm 120, a segmentation algorithm 122, and/or a path planning algorithm 124.

The method 300 comprises inserting an expandable cage between anatomical surfaces to be fused (step 304). The expandable cage may be any device configured to increase a space between two anatomical surfaces, for example to facilitate the use of one or more tools within the space. The expandable cage may utilize a mechanical, hydraulic, pneumatic, electric, electromagnetic, and/or any other type of system to generate the force needed to expand the expandable cage. The expandable cage may, in some embodiments, be a stand-alone device, while in other embodiments the expandable cage may be connected to external equipment (e.g., an external power source, an external source of pressurized air, an external fluid reservoir, etc.). In some embodiments, the expandable cage may comprise a plurality of separately controllable actuators, such that in addition to expanding a space between adjacent anatomical surfaces to be fused, the cage can facilitate moving the anatomical elements comprising those anatomical surfaces into a desired pose. One or more aspects of the expandable cage may be the same as or similar to a corresponding aspect of an interbody tool described in U.S. Patent application Ser. No. 16/927,548, filed Jul. 13, 2020 and entitled “Interbody Tool, Systems, and Methods,” the entirety of which is hereby incorporated herein by reference.

Other embodiments of the present disclosure may not use an expandable cage. For example, one or more tools may be used to increase a distance between two anatomical surfaces to be fused, and one or more rigid (e.g., non-expandable) objects may be wedged into or otherwise placed within the expanded space to maintain the increased distance between the two anatomical surfaces when the one or more tools are removed. For example, a robotically held spreader may be used to increase a distance between two pieces of a pelvic bone to be fused, and a plurality of metal rods, blocks, or other spacers may be inserted into the expanded space to maintain the increased distance between the two pieces when the spreader is removed. As another example, a rigid rod or other lever may be used to manually increase a distance between two vertebrae to be fused, after which one or more spacers may be inserted into the expanded space before the force on the lever is relaxed.

The method 300 also comprises controlling a robotic arm to prepare one or more of the anatomical surfaces to be fused within the patient using a preparation tool (step 308). The robotic arm may be, for example, a robotic arm 116, and the preparation tool may be, for example, a preparation tool 138. In some embodiments, multiple preparation tools may be used to fully prepare the anatomical surfaces for fusion. For example, one or more preparation tools may be used to cut, scrape, or otherwise detach soft tissue from one or more of the anatomical surfaces. Another one or more preparation tools may be used to sweep, brush, suction, wash away, or otherwise clear detached soft tissue and/or other anatomical material (e.g., bodily fluids, bone particles) from the one or more anatomical surfaces. Yet another one or more preparation tools may be used to perforate, roughen, or otherwise modify the one or more anatomical surfaces, to enable or facilitate successful completion of one or more subsequent aspects of the fusion process (e.g., to promote development of cellular elements deposited thereon into bone, to improve attachment between the scaffold to be printed in the step 316 and the one or more anatomical surfaces, and/or otherwise). Still another one or more preparation tools may be used to apply a chemical, surface coating, or other surface treatment to the one or more anatomical surfaces, again to enable or facilitate successful completion of one or more subsequent aspects of the fusion process, to strengthen the one or more anatomical surfaces, and/or to protect the one or more anatomical surfaces from potential harm or trauma during the fusion process.

The method 300 also comprises causing an imaging device to capture an image of an anatomical surface to be fused (step 312). The imaging may happen before preparation of one or more of the anatomical surfaces to be fused, during such preparation, after such preparation, in any combination of the foregoing, and/or at any other one or more times during the method 300. The imaging may be completed using any imaging device, including an imaging device 112. In some embodiments, the imaging device may be secured to a robotic arm and maneuvered in vitro to capture an optical, infrared, or other direct image of the one or more anatomical surfaces. The image may be analyzed—using one or more of an image processing algorithm 120 and/or a segmentation algorithm 122—to identify an area of the anatomical surface to be prepared during the step 308, to determine how to prepare the anatomical surface during the step 308 (e.g., to identify and determine a position of soft tissue attached to the anatomical surface, to determine a level of smoothness or roughness of the anatomical surface), to evaluate whether the surface has been properly prepared, to confirm that preparation of the surface is complete, and/or to identify the boundaries of the prepared surface for purposes of planning one or more aspects of one or more other steps of the method 300. Where the step 312 occurs during or after one or more of the steps 316, 320, and/or 324 of the method 300, the resulting image or images may similarly be analyzed, using one or more of an image processing algorithm 120 and/or a segmentation algorithm 122, to evaluate progress toward completion of the step in question, to aid in planning one or more aspects of the step in question, to confirm that actions taken thus far have achieved the planned and/or otherwise expected result, and/or to confirm successful completion of the step in question. Images captured during the step 312 may be used to confirm an extent of successful fixation and/or for any other purpose useful for facilitating successful completion of the method 300.

The method 300 also comprises causing a bioprinter to print a scaffold from a scaffold material, using a robotic arm to position the bioprinter (step 316). The robotic arm may be a robotic arm 116, and may be the same as or different than a robotic arm used in one or more of the steps 304, 308, and/or 312. For example, in some embodiments, one robotic arm may be configured to support a preparation tool, and a different robotic arm may be configured to support a bioprinter. In other embodiments, a single robotic arm may be operably secured to a preparation tool during or in preparation for the step 308, and may then be operably secured to a bioprinter during or in preparation for the step 316. The bioprinter may be, for example, a bioprinter 142. The bioprinter may comprise one or more internal motors or other actuators configured to move a printing head thereof relative to a base of the bioprinter. Alternatively, the bioprinter may comprise a fixed printing head, and the robotic arm secured to and/or otherwise supporting the bioprinter may be moved as needed to ensure that each drop or element of bioink is deposited in the proper location.

The bioprinter prints the scaffold out of scaffold material, which may be any polymerizable bioink. The particular bioink used to print the scaffold may be selected, for example, based on one or more properties thereof once polymerized, such as fatigue strength, shear strength, tensile strength, yield strength, toughness, wear resistance, hardness, fracture toughness, stiffness, and/or any other material property. The design of the printed scaffold may be generated, selected, or otherwise configured to yield a scaffold that will withstand forces expected to be exerted thereon during normal patient activity. For example, the printed scaffold may comprise one or more square elements, triangular elements, circular elements, intertwined elements, and/or any other element shapes or arrangements that will contribute to the scaffold having a desired strength (and/or any other property).

The scaffold may be printed in layers or other segments. The scaffold may be printed beginning at a deepest portion of an intervertebral space through which the scaffold will extend (e.g., a portion farthest from a surface incision in the patient through which the intervertebral space will be accessed) and continuing toward a shallowest portion of the intervertebral space. The scaffold may be printed starting from one of the anatomical surfaces to be fused and extending toward another of the anatomical surfaces to be fused. In some embodiments, the scaffold may be printed—at least initially—on a posterior longitudinal ligament or an anterior longitudinal ligament that extends adjacent to the intervertebral space throughout which the scaffold will extend. This may be more common, for example, when the patient is resting in a supine or prone position, respectively. Regardless of where the scaffold is initially printed, the scaffold is eventually attached to the anatomical surfaces to be fused, and extends throughout a volume positioned between or among the anatomical surfaces to be fused.

The method 300 also comprises causing a polymerization tool to induce polymerization of the scaffold material, using a robotic arm to position the polymerization tool (step 320). The polymerization tool may be configured to spray, squirt, dispense, or otherwise apply an enzyme or other chemical to the scaffold material to induce polymerization thereof. Alternatively, the polymerization tool may be configured to emit light, ultrasound, or any other form of energy onto the scaffold material to induce polymerization thereof. The polymerization tool may be selected based on the particular bioink used to print the scaffold, or vice versa. In other words, the particular polymerization tool used for the step 320 must utilize an enzyme or other chemical or type of energy that will induce polymerization of the particular scaffold material used to print the scaffold.

The polymerization tool may in some embodiments be carefully controlled to induce polymerization only of scaffolding material that falls within a specific volume within the intervertebral space. Use of an accurate robotic arm to control the polymerization tool may facilitate precise control of the polymerization process, which may also be guided and/or otherwise assisted by imaging and/or navigation. In some embodiments, the polymerization tool may be carefully controlled to induce polymerization only of scaffolding material that is within an expected scaffold volume. In other words, if the scaffold design includes a linear element with a precise boundary, and during printing of that scaffold element some bioink was deposited or slipped or otherwise became located outside of the precise boundary, then the polymerization tool may be configured to induce polymerization only of the bioink within the precise boundary (e.g., by controlling emission of the energy or application of the enzyme or other chemical). Any scaffold material that is not polymerized may be washed away, suctioned, or otherwise removed from the intervertebral space at some point during the operation, or may be cleaned through normal biological processes. In this way, a final, polymerized scaffold may be obtained that closely matches the intended design thereof.

In some embodiments of the method 300, the steps 316 and 320 (and/or 324) may happen iteratively or simultaneously. For example, the bioprinter may be caused to print a single layer of the scaffold, after which the polymerization tool may be used to induce polymerization of only the scaffold material in that layer (and, in some embodiments, an impregnation tool may be used to inject cellular elements into the polymerized scaffold material, as described in more detail below). The bioprinter may then be caused to print another layer of the scaffold, which layer may then be polymerized before the next layer is printed, and so on. Such iterative printing and polymerization may occur on a level-by-level basis, an element-by-element basis, a segment-by-segment basis, or on any other basis. Moreover, such iterative printing, polymerization, and impregnation may enable the creation of a structure comprising a plurality of closed or substantially closed pockets, each filled with cellular elements. Such a design may contribute to faster bone growth and/or higher strength than a scaffold design that has larger, open spaces filled with cellular elements.

In some embodiments, the same robotic arm may be used to manipulate both the bioprinter and the polymerization tool (e.g., may first be secured to the bioprinter, and then to the polymerization tool, and then to the bioprinter again, and so forth). In other embodiments, a first robotic arm may be used to manipulate the bioprinter, and a second robotic arm may be used to manipulate the polymerization tool, such that neither robotic arm needs to switch tools.

With two robotic arms holding the bioprinter and the polymerization tool, respectively, the printing and polymerization steps may occur simultaneously. In other words, as the bioprinter prints a portion of the scaffold, the polymerization tool may be used to immediately induce polymerization of that portion of the scaffold (or of another recently printed portion of the scaffold). In this way, the scaffold can be polymerized as it is printed, rather than waiting for the entire scaffold to be printed before beginning polymerization. Iterative or simultaneous printing and polymerization of the scaffold may further ensure that the scaffold retains its printed shape (as polymerization causes the scaffold material to stiffen), which may not occur if the entire scaffold is first printed and then induced to polymerize.

Also in some embodiments, the polymerization tool may not be controlled or manipulated by a robotic arm. For example, a polymerization tool may be an ultrasound positioned external to the patient and secured to a frame or other support. Such a polymerization tool may be configured with an adjustable aperture or other mechanism that enables the tool to adjust a direction in which energy is emitted, a beam width of any emitted energy, and/or one or more other characteristics to ensure that polymerization of scaffold material occurs only where desired.

The method 300 also comprises causing an impregnation tool to impregnate the scaffold with cellular elements, using a robotic arm to position the impregnation tool (step 324). The impregnation tool may be an impregnation tool 150 or any other impregnation tool. The robotic arm may be, for example, a robotic arm 116, and may be the same robotic arm used in one or more of the steps 308, 312, 316, and/or 320, or a different robotic arm. The impregnation tool is designed to deliver cellular elements in any useful way to the intervertebral space for impregnation of the scaffold. For example, the impregnation tool may be designed to discharge, spray, apply, inject, pump, squeeze, or otherwise transfer cellular elements from a reservoir or channel of the impregnation tool and into the intervertebral space and/or onto the scaffold. Impregnating the scaffold with cellular elements may comprise forcing cellular elements into interstitial spaces between or among elements of scaffold material, and/or filling the remainder of a volume partially occupied by the scaffold with the cellular elements. In a given volume occupied by the scaffold and the cellular elements, the scaffold occupies a minority of the volume, and the cellular elements occupy a majority of that volume.

The cellular elements may be or comprise bone graft material, which may be or include osteoblast cells, osteocyte cells, and/or osteoclast cells. In some embodiments, the cellular elements may comprise crushed bone or other bone material, whether from the patient (e.g., autograft) or from a bone donor (e.g., allograft). The cellular elements may be or comprise natural elements and/or synthetic elements. The cellular elements may be any cellular elements useful for causing and/or promoting bone growth. The cellular elements may be or comprise any material identified or disclosed in Ashammakhi et al., “Advancing Frontiers in Bone Bioprinting,” Advanced Health Care Materials, at 8 (Wiley-VCH Verlag GmbH & Co. 2019), the entirety of which is hereby incorporated by reference herein.

As with the printing and polymerization steps, the impregnation step may happen iteratively and/or simultaneously with one or more other steps of the method 300. For example, cellular elements may be impregnated in the scaffold structure on a layer-by-layer or other iterative basis as the scaffold is printed and polymerized. As another example, cellular elements may be continuously impregnated in the scaffold as the scaffold is being printed and polymerized.

The method 300 also comprises removing the expandable cage (step 328). Once the fusion structure, comprising the polymerized scaffold impregnated with cellular elements, is complete, the expandable cage (or other spacers or spacing elements) may be removed from between or among the anatomical surfaces to be fused. With the expandable cage or other spacing elements gone, the fusion structure remains in force-transmitting communication with the anatomical surfaces at issue, and transmits forces therebetween. Although bone growth within the fusion structure will take some time, the scaffold of the fusion structure is sufficiently strong, at least in some embodiments, to withstand forces exerted thereon during normal activities of the patient (e.g., sitting, standing, walking, and other non-strenuous activity).

The present disclosure encompasses embodiments of the method 300 that comprise more or fewer steps than those described above, and/or one or more steps that are different than the steps described above.

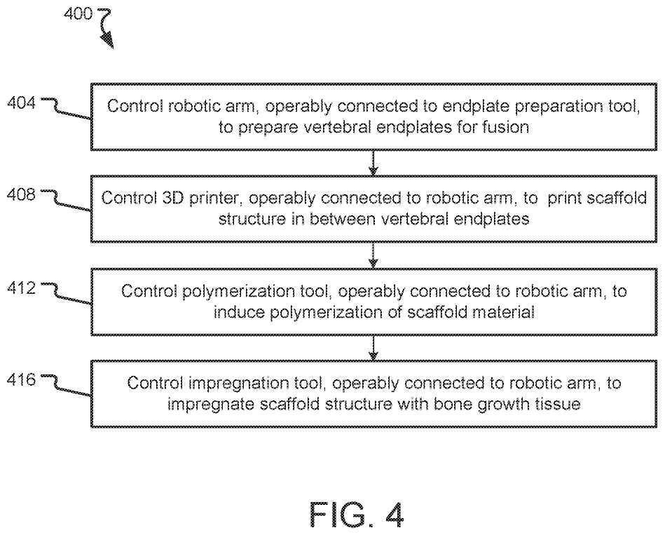

FIG. 4 depicts a method 400 that may be used, for example, to achieve spinal fusion. One or more aspects of the method 400 may be used independently and/or together with one or more aspects of any other method described herein according to embodiments of the present disclosure.

The method 400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 400. The at least one processor may perform the method 400 by executing instructions (e.g., instructions 126) stored in a memory such as the memory 106. The instructions may correspond to one or more steps of the method 300 described below. The instructions may cause the processor to execute one or more algorithms, such as an image processing algorithm 120, a segmentation algorithm 122, and/or a path planning algorithm 124.

The method 400 comprises controlling a robotic arm, operably connected to an endplate preparation tool, to prepare vertebral endplates for fusion (step 404). The robotic arm may be a robotic arm 116 or any other robotic arm, and may be holding (e.g., via an end effector), attached to, or otherwise supporting the endplate preparation tool. The endplate preparation tool may be any one or more preparation tools 138 or other surface preparation tools. The step 404 may comprise controlling the robotic arm to use the endplate preparation tool to scrape soft tissue from the vertebral endplates, remove the soft tissue from an intervertebral space between the endplates, clean the vertebral endplates, modify the vertebral endplates so as to promote bone growth thereon (e.g., by perforation thereof or otherwise), and/or apply one or more chemicals or other substances to the vertebral endplates to facilitate attachment of a scaffold structure thereto, to facilitate bone growth thereon, to strengthen the vertebral endplates, or to achieve any other clinical purpose.

In some embodiments, a thickness or other characteristic of a coating applied to the vertebral endplates may have tight tolerances. In such embodiments, the endplate preparation tool used to apply the coating to the vertebral endplate may be configured to apply the coating within the specified tolerances, and may further comprise a sensor or other device or tool for measuring the characteristic in question or otherwise confirming compliance with the specified tolerances.

The method 400 also comprises controlling a 3D printer, operably connected to a robotic arm, to print a scaffold structure in between the vertebral endplates (step 408). One or more aspects of the step 408 may be the same as or similar to one or more aspects of the step 316 of the method 300. The robotic arm may be the same robotic arm as in the step 404, or a different robotic arm. The robotic arm may be a robotic arm 116. The 3D printer may be a bioprinter 142 or any other printer useful for printing using bioink. The printer may be held by or otherwise secured to the robotic arm, and may comprise a movable printing head capable of printing the scaffold structure without movement of the robotic arm, or may rely on the robotic arm for proper positioning of the printing head. The scaffold structure may be any scaffold structure extending between the two vertebral endplates. A design of the scaffold structure may be predetermined and/or selected based on one or more properties of the scaffold structure, including, for example, ability of the scaffold structure (once complete) to withstand forces that may be imposed thereon by the vertebrae associated with the vertebral endplates to which the scaffold structure is attached. The scaffold structure may extend throughout the intervertebral space between the vertebral endplates, and may or may not extend to a perimeter of the intervertebral space.

The method 400 also comprises controlling a polymerization tool, operably connected to a robotic arm, to induce polymerization of the scaffold material (step 412). The step 412 may be the same as or similar to the step 320 of the method 300. Moreover, the steps 408 and 412 may occur iteratively or simultaneously, in the same manner as or in a similar manner to the manner described above in connection with the steps 316 and 320 of the method 300.

The method 400 also comprises controlling an impregnation tool, operably connected to a robotic arm, to impregnate the scaffold structure with bone growth tissue (step 416). The step 416 may also occur in the same manner as or in a similar manner to the step 324 of the method 300, and may occur after, iteratively with, or simultaneously with one or more of the steps 408 and 412, just as the step 324 may occur after, iteratively with, or simultaneously with one or more other steps of the method 300.

Throughout the method 400, the same robotic arm may be used for each step, or different robotic arms may be used for one or more steps. Any steps of the methods 300 and 400 described above as utilizing a robotic arm may involve use of a path planning algorithm 124 or other algorithm useful for determining how to manipulate the robotic arm to place a preparation tool 138, bioprinter 142, polymerization tool 146, impregnation tool 150, imaging device 112, and/or any other tool or device in a desired or predetermined pose.

The present disclosure encompasses embodiments of the method 400 that comprise more or fewer steps than those described above, and/or one or more steps that are different than the steps described above.

As noted above, the present disclosure encompasses methods with fewer than all of the steps identified in FIGS. 3 and 4 (and the corresponding description of the methods 300 and 400), as well as methods that include additional steps beyond those identified in FIGS. 3 and 4 (and the corresponding description of the methods 300 and 400). The present disclosure also encompasses methods that comprise one or more steps from one method described herein, and one or more steps from another method described herein. Any correlation described herein may be or comprise a registration or any other correlation.

Any aspect of the methods 300 and/or 400 may be the same as or similar to any corresponding aspect of the description of FIGS. 2A-21 above, and vice versa. The use of FIGS. 2A-2I to provide one illustration of embodiments of the present disclosure, and the use of FIGS. 3 and 4 to provide additional illustrations of embodiments of the present disclosure, should not be understood to mean that any aspect of any described embodiment is applicable only to that particular embodiment.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An in-situ fusion system, comprising:
at least one robotic arm;
a bioprinter;
a polymerization tool;
at least one processor; and
a memory storing instructions for execution by the at least one processor that, when executed, cause the at least one processor to:
control the at least one robotic arm to prepare at least two bone surfaces to support cellular growth;
cause the bioprinter to print, from a scaffold material, a scaffold between the at least two bone surfaces; and
cause the polymerization tool to induce the scaffold material to polymerize.

2. The system of claim 1, further comprising:
a cellular impregnation tool;
wherein the memory stores additional instructions for execution by the at least one processor that, when executed, cause the at least one processor to:
cause the cellular impregnation tool to impregnate the scaffold with cellular elements, using a robotic arm of the at least one robotic arm to position the cellular impregnation tool.

3. The system of claim 1, wherein controlling the at least one robotic arm to prepare the at least two bone surfaces to support cellular growth comprises controlling the at least one robotic arm to:
clean the at least two bone surfaces; and
apply a surface treatment to each of the at least two bone surfaces.

4. The system of claim 1, wherein the memory stores additional instructions for execution by the at least one processor that, when executed, cause the at least one processor to:
repeat the causing the bioprinter to print the scaffold and the causing the polymerization tool to induce the scaffold material to polymerize until the scaffold extends from one of the at least two bone surfaces to another of the at least two bone surfaces.

5. The system of claim 1, wherein the polymerization tool is configured to apply energy to the scaffold material to induce the scaffold material to polymerize.

6. The system of claim 5, wherein the polymerization tool is configured to apply an enzyme to the scaffold material to induce the scaffold material to polymerize.

7. The system of claim 1, wherein the memory stores additional instructions for execution by the at least one processor that, when executed, cause the at least one processor to:
insert an expandable cage between the at least two bone surfaces to hold the at least two bone surfaces in a desired position.

8. The system of claim 7, wherein the causing the bioprinter to print a scaffold between the at least two bone surfaces and the causing the polymerization tool to induce the scaffold material to polymerize occur simultaneously.

9. The system of claim 1, wherein each of the bioprinter and the polymerization tool is selectively attachable to the at least one robotic arm.

10. The system of claim 1, wherein the at least one robotic arm comprises a single robotic arm, and further wherein the single robotic arm is used to position the bioprinter for printing the scaffold and to position the polymerization tool for inducing the scaffold material to polymerize.

11. A robotic surgical system comprising:
a robotic arm selectively connectable to each of a preparation tool, a printing tool, and a cellular impregnation tool;
at least one processor; and
a memory storing instructions for execution by the at least one processor that, when executed, cause the at least one processor to:
cause the robotic arm to use the preparation tool to prepare an anatomical surface inside a patient for bone growth thereon;
cause the robotic arm to use the printing tool to print a scaffold inside the patient that connects to the anatomical surface; and
cause the robotic arm to use the cellular impregnation tool to impregnate the scaffold with bone tissue cells.

12. The system of claim 11, wherein preparing the anatomical surface comprises causing the robotic arm to use the preparation tool to create a plurality of holes in the anatomical surface.

13. The system of claim 11, wherein the scaffold is printed and impregnated with bone tissue cells one layer at a time.

14. The system of claim 13, wherein the anatomical surface is a vertebral endplate; the scaffold, when finished, connects the vertebral endplate with an opposite vertebral endplate; and a first layer of the scaffold is printed on an anterior ligament.

15. The system of claim 11, wherein impregnating the scaffold with bone tissue cells comprises filling a volume defined by the scaffold with bone tissue cells.

16. The system of claim 11, further comprising an imaging device, and wherein the memory stores additional instructions for execution by the at least one processor that, when executed, further cause the at least one processor to:
cause the imaging device to capture an image of the anatomical surface after the anatomical surface has been prepared for bone growth thereon.

17. An in-situ vertebral fusion method comprising:
controlling a 3D printer, operably connected to a robotic arm, to print, in between two vertebral endplates and using a polymerizable scaffold material, a scaffold structure; and
controlling a polymerization tool, operably connected to the robotic arm, to induce polymerization of the scaffold material.

18. The method of claim 17, further comprising:
controlling an impregnation tool, operably connected to the robotic arm, to impregnate the scaffold structure with bone growth tissue.

19. The method of claim 17, further comprising:
controlling the robotic arm, operably connected to an endplate preparation tool, to prepare each of the two vertebral endplates for bone growth thereon.

20. The method of claim 17, wherein controlling the robotic arm to prepare each of the two vertebral endplates for bone growth thereon comprises controlling the robotic arm to clean each of the two vertebral endplates and to apply a surface treatment to each of the two vertebral endplates.
